(19) United States
(12) Patent Application Publication
(10) Pub. No.: US 2023/0080695 A1
(43) Pub. Date: Mar. 16, 2023

(54) LACTOFERRIN FOR ORAL USE WITH ANTIVIRAL ACTION

(71) Applicant: SOFAR S.P.A., Trezzano Rosa (MI) (IT)

(72) Inventors: Andrea BIFFI, Trezzano Rosa (MI) (IT); Walter FIORE, Trezzano Rosa (MI) (IT)

(21) Appl. No.: 17/798,050

(22) PCT Filed: Oct. 15, 2020
(86) PCT No.: PCT/IB2020/059695

§ 371 Date: Aug. 5, 2022

(30) Foreign Application Priority Data
Mar. 9, 2020 (IT) 102020000005011

(51) Int. Cl. A61K 38/40; A61P 31/14; A61K 9/127

(52) U.S. Cl. CPC A61K 38/40; A61P 31/14; A61K 9/1271

(57) ABSTRACT

The present invention relates to a composition comprising lactoferrin for oral use as an antiviral, preferably for use in the treatment of viral infections of the respiratory system and of symptoms or disorders deriving from, or relating to, said viral infections, preferably SARS-coronavirus viral infections (e.g. COVID-19).

The present invention relates to a composition comprising lactoferrin for oral use as an antiviral, preferably for use in the treatment of viral infections of the respiratory system and of symptoms or disorders deriving from, or relating to, said viral infections, preferably SARS-coronavirus viral infections (e.g. COVID-19).

Viral infections of the respiratory tract, as the name says, are infectious diseases caused by viruses that affect the organs of the upper and/or lower respiratory system (nose, pharynx, larynx, trachea, bronchi and lungs).

Preferably, the present invention relates to viral infections caused by at least one virus of the severe acute respiratory syndrome coronavirus species, abbreviated as SARS-CoV. Said viruses of the SARS-CoV species are positive-strand RNA viruses (group IV of the Baltimore classification), belonging to the genus of Betacoronavirus.

A virus of the severe acute respiratory syndrome coronavirus species is the virus that caused the 2002-2003 SARS epidemic in China, called SARS-CoV strain.

It was first discovered in November 2002 in the Chinese province of Guangdong. From Nov. 1, 2002 to Aug. 31, 2003, the virus infected 8,096 people in about thirty countries, causing 774 deaths, mainly in China, Hong Kong, Taiwan and all of Southeast Asia. Toward the end of 2019, a second virus of the severe acute respiratory syndrome coronavirus species, called SARS-CoV-2 strain or, alternatively, 2019-nCoV, caused a new SARS epidemic in China and in the rest of the world, more commonly known as COVID-19 (COronaVIrus Disease 19, also known as SARS-CoV-2 acute respiratory disease or coronavirus disease 2019, also coronavirus syndrome 2019).

Following an extensive research and development activity, the Applicant, addresses and solves the problem of the treatment of viral infections, preferably viral infections of the respiratory tract (upper and lower respiratory tract), in particular, viral infections of the respiratory tract caused by at least one virus of the severe acute respiratory syndrome coronavirus species (such as SARS-CoV, SARS-CoV-2/2019-nCoV strain—whose disease is known as COVID-19—or SARS-CoV-like), by providing compositions for oral use comprising lactoferrin or a derivative thereof for use in methods for the treatment of said viral infections or symptoms or disorders related thereto.

Lactoferrin, also known as lactotransferrin, is a multifunctional globular protein. Lactoferrin belongs to the transferrin family and it has a molecular mass of about 80 KDa, with two binding sites for the ferric ion (Fe³⁺), similarly to the transferrin itself. Lactoferrin is never saturated with iron and its ferric content varies. Lactoferrin has antimicrobial activity, bactericidal, fungicidal and against various viruses. It is hypothesised that the antimicrobial activity of lactoferrin is related to its affinity for Fe³⁺, therefore to its high ability to compete in the free state with iron-dependent microorganisms, and to a direct action on the external membrane of Gram-negative bacteria. The combination of lactoferrin with ferric ion in mucosal secretions modulates the activity and aggregative ability of bacteria and viruses toward cell membranes. This is due to the fact that some bacteria and viruses require iron in order to carry out cell replication and lactoferrin, on the contrary, removes it from the surrounding environment, preventing the proliferation of said bacteria and viruses.

Lactoferrin exhibits antiviral activity against DNA and RNA viruses, including rotavirus, respiratory syncytial virus, herpes virus and HIV. The antiviral effect of lactoferrin lies in the early stage of infection. Lactoferrin prevents the virus from entering into the host cell by blocking cell receptors or binding directly to virus particles. Specifically, the antiviral effect of lactoferrin mainly lies in its ability to bind to glycosaminoglycans of the plasma membrane. Furthermore, it is known in the literature that lactoferrin participates in the host's immune response against acute invasion of severe acute respiratory syndrome coronavirus (SARS-CoV) by improving NK cell activity and by stimulating neutrophil aggregation and adhesion. Furthermore, it has been hypothesised that lactoferrin can play a protective role in the host's defence against SARS-CoV infection by binding to HSPGs (HSPG, heparan sulfate proteoglycans, widely distributed) and by blocking the preliminary interaction between SARS-CoV and host cells, given that HSPGs are essential molecules of the cell surface involved in the entry of SARS-CoV cells.

In the context of the present invention, the expression lactoferrin derivatives is used to indicate any multifunctional peptide or globular protein deriving from lactoferrin which shows similar antiviral effects, for example apolactoferrin or lactoferricin. Lactoferricin is a lactoferrin derivative with known antibacterial activity, apolactoferrin is lactoferrin in which the N-terminal lobe (or apolactoferrin) takes an open conformation.

The compositions of the invention, based on lactoferrin or a derivative thereof, formulated for oral use, preferably in solid form, are effective as antivirals, in particular in the treatment of viral infections of the respiratory tract and of the symptoms or disorders related thereto, in particular, infections caused by at least one virus of the severe acute respiratory syndrome coronavirus species (such as SARS-CoV, SARS-CoV-2 or 2019-nCoV strains—responsible for the disease known as COVID-19—or SARS-CoV-like).

The compositions of the invention, based on lactoferrin or a derivative thereof, can be formulated, by adding specific excipients and additives, as solutions or emulsions or dispersions suitable to be atomised and administered—using a spray device—into the nose and throat for inhalation, oral or nasal use. Said sprayable compositions are effective as antivirals, in particular in the treatment of viral infections of the respiratory tract and of the symptoms or disorders related thereto in particular infections caused by at least one virus of the severe acute respiratory syndrome coronavirus species (such as SARS-CoV, SARS-CoV-2 or 2019-nCoV strain—responsible for the disease known as COVID-19—or SARS-CoV-like).

The compositions of the invention, based on lactoferrin or a derivative thereof, have no relevant side effects and they can be administered to all categories of subjects in need, including the elderly, pregnant or breastfeeding women, paediatric subjects (0-12 years), subjects with respiratory or cardiovascular complications or diabetes or other complications that may pose a risk or danger in the event of a viral infection.

Furthermore, the compositions of the invention, based on lactoferrin, are easy to prepare and cost-effective.

These and other objects, which will be clear from the detailed description that follows, are attained by the compositions and the mixtures of the present invention due to the technical characteristics reported in the description and claimed in the attached claims.

### DESCRIPTION OF THE FIGURES

FIGS. 1A-D represent the effect of lactoferrin (L) on a panel of cytokines/chemokines and molecules with antiviral action or involved in antiviral responses produced by Caco-2 intestinal epithelial cells.

FIGS. 2A and 2B schematically represent the drawings of the in vitro study of evaluation of the antiviral responses in Caco-2 intestinal epithelial cells following a pre-treatment or co-treatment with lactoferrin (L) with respect to a treatment with SARS-CoV-2 virus.

FIGS. 3A-E represent the effect of lactoferrin on a panel of cytokines/chemokines and molecules with antiviral action or involved in antiviral responses produced by Caco-2 intestinal epithelial cells following a pre-treatment with lactoferrin (L) with respect to a treatment with SARS-CoV-2 virus.

FIGS. 4A-D represent the effect of lactoferrin on a panel of cytokines/chemokines and molecules with antiviral action or involved in the antiviral responses produced by Caco-2 intestinal epithelial cells following a co-treatment with lactoferrin (L) and with SARS-CoV-2 virus.

### DETAILED DESCRIPTION OF THE INVENTION

Forming an object of the present invention is a composition for oral use (in short, composition of the invention) for use as an antiviral, preferably for use in a method for the treatment of viral infections of the respiratory system (upper respiratory tract and/or lower respiratory tract) and symptoms or disorders deriving from or relating to said viral infection in subjects in need, wherein said composition comprises: (i) a mixture M (in short, mixture M of the invention) comprising or, alternatively, consisting of lactoferrin (in short, LF) or a derivative thereof of an acceptable pharmaceutical grade; and, optionally, (ii) at least one acceptable pharmaceutical grade additive and/or excipient.

Preferably, the viral infection treated using the composition of the invention is an infection caused by a virus of the family Coronaviridae, subfamily: Coronavirinae, genus: Betacoronavirus, species: severe acute respiratory syndrome coronavirus (in short, SARS-CoV or SARS-coronavirus); selected from the following strains: (I) severe acute respiratory syndrome coronavirus (SARS-CoV or SARS) (II) severe acute respiratory syndrome coronavirus-2 (SARS-CoV-2 or 2019-nCoV—responsible for the disease known as COVID-19—), and (Ill) severe acute respiratory syndrome coronavirus-like (SARS-CoV-like or SL-CoV); preferably SARS-CoV-2 or 2019-nCoV, responsible for the disease known as COVID-19.

In short, in the context of the present invention these viruses (e.g. (I), (II) and (Ill)) are referred to as “virus of the SARS-coronavirus species” or simply “SARS-coronavirus”.

Symptoms or disorders deriving from or related to said viral infection of the respiratory tract (upper respiratory tract and/or lower respiratory tract), preferably a coronavirus infection as defined above (e.g. SARS-CoV, SARS-CoV-2 or 2019-nCoV, SARS-CoV-like) can be: severe acute respiratory syndrome (SARS), respiratory complications, asthma, chronic obstructive pulmonary disease (COPD), bronchitis, emphysema, cystic fibrosis, cough, pertussis, pneumonia, pleurisy, bronchiolitis, cold, sinusitis, rhinitis, tracheitis, pharyngitis, laryngitis, acute laryngotracheobronchitis, epiglottitis, bronchiectasis, difficulty breathing, dyspnoea (breathlessness, shortness of breath) fever, fatigue, muscle ache and/or pain, nasal congestion, runny nose, sore throat, gastrointestinal symptoms such as for example nausea and diarrhoea, kidney failure, loss of appetite and/or general feeling unwell.

Lactoferrin may be present in the compositions of the invention or in the mixtures M of the invention at a % by weight from 10% to 90% with respect to the total weight of the composition or of the mixture M (for example, 15%, 20%, 25%, 30%, 35%, 40%, 45%, 50%, 55%, 60%, 65%, 70%, 75%, 80%, or 85%), preferably from 20% to 80%, more preferably from 30% to 70% or from 30% to 50%.

The composition of the invention, comprising said mixture M according to any one of the embodiments of the present invention, may further comprise said at least one pharmaceutical or food grade additive and/or excipient, i.e. a substance devoid of therapeutic activity suitable for pharmaceutical or food use. In the context of the present invention the additives and/or excipients acceptable for pharmaceutical or food use comprise all ancillary substances known to the man skilled in the art for the preparation of compositions in solid, semi-solid or liquid form, such as for example diluents, solvents (including water, glycerine, ethyl alcohol), solubilisers, acidifiers, thickeners, sweeteners, flavour enhancers, colouring agents, lubricants, surfactants, preservatives, stabilisers, pH stabilising buffers and mixtures thereof.

The composition for oral use of the present invention may be formulated in a solid form selected from: tablets, chewable tablets, oral soluble tablets, granules, powder, flakes, soluble powder or granules, oral soluble powder or granules, capsules; or, alternatively, in liquid form selected from: solutions, suspensions, dispersions, emulsions, liquid which can be dispensed in spray form, syrups; or, alternatively, in semi-liquid form selected from: soft-gel, gel; preferably the composition of the invention is in solid form.

In the mixture M of a composition of the invention, according to any one of the embodiments described in the present invention, lactoferrin may be in a liposomal form, for example phospholipid-based liposomal form.

Said liposomal form (or formulation) of lactoferrin may reduce the clearance of lactoferrin after administration (oral or intra-nasal by means of spray formulation) and, therefore, increase the degree of absorption thereof. In addition, the substances carried by the liposomes are protected against the action of enzymes (proteases, nucleases) or denaturing environments (pH). Liposomes are hollow microspheres formed by one or more lipid bilayers, whose membrane generally consists of cholesterol (or cholesterol esters) and phospholipids such as phosphatidylcholine, diacetyl phosphate, and phosphatidylethanolamine. The liposomes have dimensions that may vary from 20 to 25 nm, up to 2.5 μm. In the context of the present invention, the term for oral use is used to indicate both oral (or gastroenteric) administration and sublingual (or buccal) administration.

The composition of the invention for oral use, preferably in solid form, is effective as an antiviral, in particular in the treatment of respiratory tract infections caused by a SARS-coronavirus virus, preferably SARS-CoV or 2019-nCoV, responsible for the disease known as COVID-19, in daily doses of lactoferrin comprised in the range from 5 mg to 1000 mg, preferably from 10 mg to 500 mg, more preferably from 20 mg to 400 mg, for example from 50 mg to 350 mg, from 50 mg to 300 mg, from 50 mg to 250 mg, from 50 mg to 200 mg, from 100 mg to 200 mg.

The aforementioned daily doses can be administered to the subject in need in a single dose (one dose) or in repeated doses, for example two, three or four daily doses.

The compositions of the invention, according to any of the described embodiments, may be for use as adjuvants of further antiviral therapeutic approaches.

Unless specified otherwise, the expression composition or mixture or other comprising a component at an amount “comprised in a range from x to y” is used to indicate that said component can be present in the composition or mixture or other at all the amounts present in said range, even though not specified, extremes of the range comprised.

Unless specified otherwise, the indication that a composition or mixture “comprises” one or more components or substances means that other components or substances can be present besides the one, or the ones, indicated specifically.

In the context of the present invention, the expression “treatment method” is used to indicate an intervention on a subject in need, comprising the administration of a therapeutically effective amount (according to a man skilled in the art) of a composition or mixture of substances with the aim of eliminating, reducing/decreasing or preventing a disease or ailment and symptoms or disorders thereof. In the context of the present invention, the term “subject/s” is used to indicate human or animal subjects, preferably mammals (e.g. pets such as dogs, cats, horses, sheep or cattle). Preferably, the compositions of the invention are for use in treatment methods for human subjects.

Preferred embodiments of the present invention FRn are reported below.

FR1. A composition for use in a method for the treatment of a viral infection,

wherein said composition comprises
(i) lactoferrin; and, optionally,
(ii) at least one acceptable pharmaceutical grade additive and/or excipient; and

wherein said composition is for use through oral route.

FR2. A composition for use according to FR1, wherein said composition is for use in a method for the treatment of a viral infection of the respiratory system and of symptoms and/or disorders deriving from or relating to said viral infection; preferably viral infections of the upper respiratory tract and/or of the lower respiratory tract.

FR3. The composition for use according to FR1 or FR2, wherein said viral infection is caused by a virus of the family Coronaviridae, subfamily: Coronavirinae, genus: Betacoronavirus, species: severe acute respiratory syndrome coronavirus, selected from strains: severe acute respiratory syndrome coronavirus (SARS-CoV), severe acute respiratory syndrome coronavirus-2 (SARS-CoV-2 or 2019-nCoV) and responsible for COVID-19 disease, and severe acute respiratory syndrome coronavirus-like (SARS-CoV-like or SL-CoV); preferably SARS-CoV-2.

FR4. The composition for use according to FR2 or FR3, wherein said symptoms and/or disorders deriving from or relating to said viral infection of the respiratory system are selected from: severe acute respiratory syndrome (SARS), respiratory complications, asthma, chronic obstructive pulmonary disease (COPD), bronchitis, emphysema, cystic fibrosis, cough, pertussis, pneumonia, pleurisy, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, kidney failure, loss of appetite, general feeling unwell.

FR5. The composition for use according to any one of the preceding FRns, wherein the composition is in solid form selected from: tablets, chewable tablets, oral soluble tablets, granules, powder, flakes, soluble powder or granules, oral soluble powder or granules, capsules; or, alternatively, in liquid form selected from: solutions, suspensions, dispersions, emulsions, liquid which can be dispensed in spray form, syrups; or, alternatively, in semi-liquid form selected from: soft-gel, gel; preferably in solid form.

FR6. The composition for use according to any one of the preceding FRns, wherein lactoferrin is in a liposomal form; preferably in a phospholipid-based liposomal form.

### Experimental Part

The Applicant carried out in vitro studies in order to evaluate the ability of lactoferrin to stimulate the innate antiviral immune response in a subject in order to fight SARS-CoV-2 (COVID-19) virus infection. In detail, the following were evaluated:

(1) the ability of lactoferrin to enhance antiviral responses in intestinal epithelial cells; and
(2) the ability of lactoferrin to influence SARS-CoV-2 infection in human intestinal epithelial cells.

### Materials (1) and (2)

Confluent monolavers of Caco-2 cells (immortalized epithelial cells from colon carcinoma): Caco-2 cells were obtained from the European Collection of Authenticated Cell Cultures (ECACC), and grown in T25 bottles in complete DMEM medium (Dulbecco's Modified Eagle Medium supplemented with 10% (v/v) foetal bovine serum (FBS), 1% (v/v) sodium pyruvate and 1% (v/v) penicillin and streptomycin) at 370 in humidified incubator containing 5% CO₂, separated upon reaching a confluence of about 75% and seeded to a concentration of 5×10⁵ cells/well in 12 wells/plates. The culture medium was changed every 48 hours until the cells formed a confluent monolayer. Then, the culture medium was removed and replaced with a fresh medium without antibiotics.
Lactoferrin (code “L”) (100 μg/mL).
SARS-CoV-2 Virus (code, “SARS”): it was isolated from a patient in Padua and completely characterised; the viruses were propagated on VERO C1008 cells, to obtain a working stock which was stored at −80° C.

### 1. Effect of Lactoferrin on Antiviral Responses in Intestinal Epithelial Cells.

### 1.1. Method

1) Confluent monolayers of Caco-2 cells were incubated for 2 hours only with culture medium (control, code “nt”: not treated) or with lactoferrin (code “L”).

2) After 4 hours, the culture medium was removed, replaced with a complete medium containing antibiotics and the monolayers were incubated for another 16 hours at 37° C.

3) At the end of incubation, the medium was removed, the monolayers were washed with frozen DMEM, the cells were collected and immediately lysed. Total RNA was extracted from the cells, transcribed into cDNA (iScript™ Select cDNA Synthesis Kit (BioRad)) and used to perform quantitative RT-PCR on the following mRNA-transcriptors:
IFN-β and IFN-α, specific for antiviral action;
IRF-3 and IRF-7, signalling molecules linked to interferons (antiviral molecules);
TLR3 and TLR7, specific receptors for innate immunity, which “sees” viral RNA and initiates the antiviral response;
IL-10 and TGF-β, anti-inflammatory markers;
IL6, pro-inflammatory marker
4) All tests were conducted three times. Housekeeping gene Rn18S was used as reference. The data were analysed using the ΔΔCt (fold change) method.

### 1.2. Results

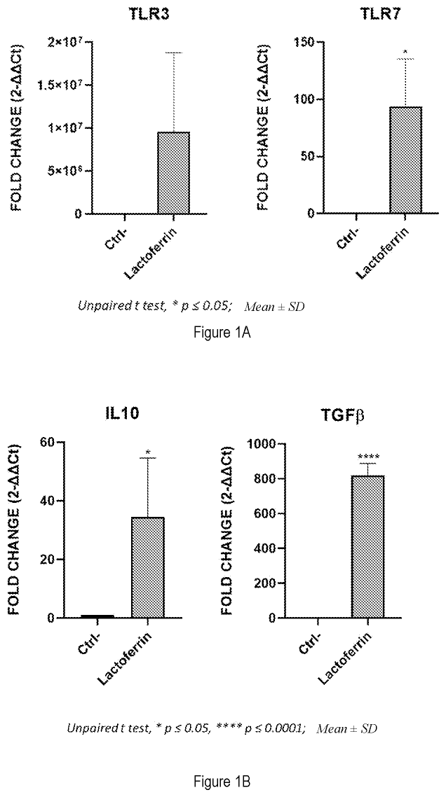

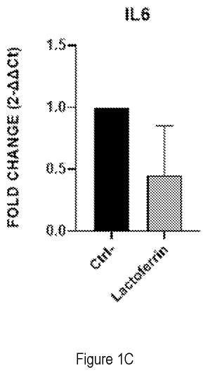

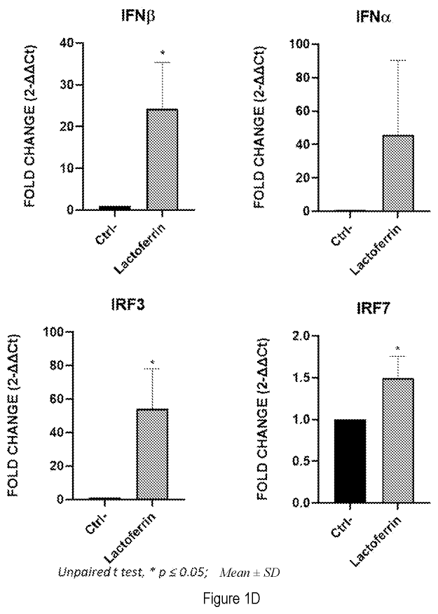

Lactoferrin is capable of stimulating the innate immune defenses (TLR3 and TLR7; FIG. 1A), exerting a good anti-inflammatory response (significant data on IL-10, TGF-β; FIG. 1B) and with a tendency to reduce the pro-inflammatory markers (IL6; FIG. 1C). Furthermore, lactoferrin showed significant activity on several signal pathways involved in the antiviral response (IFN-β, IFN-α, IRF-3 and IRF-7; FIG. 1D). Expression of the genes encoding the α and β interferons (IFN-α and IFN-β) was quantified as signalling proteins released from host cells in response to the presence of the virus. The human intestinal mucosa produces these molecules to fight virus infection.

Other cytokines were quantified in terms of gene expression: IL-10 and TGF-β, which are anti-inflammatory markers; stimulation of the expression of anti-inflammatory cytokines is a valuable tool for counterbalancing the deleterious effects of viral infection.

Furthermore, TLR3 and TRL7 receptors are involved in the reaction to the virus through the recognition of single and double stranded RNA; generally, the activation of TLR predisposes to the release of interferons.

### 2. Effect of Lactoferrin on SARS-CoV-2 Infection in Human Intestinal Epithelial Cells

### 2.1. Method

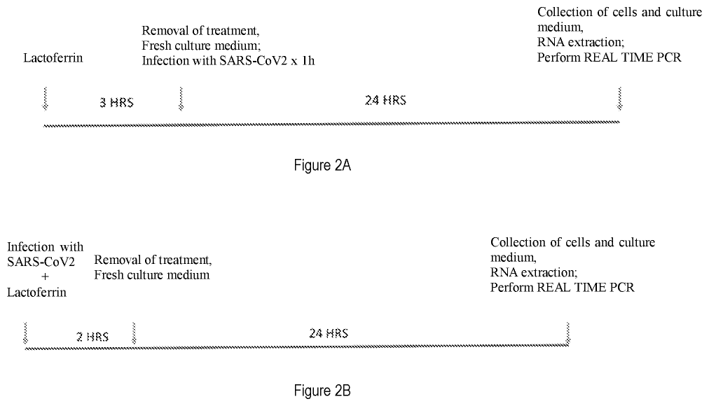

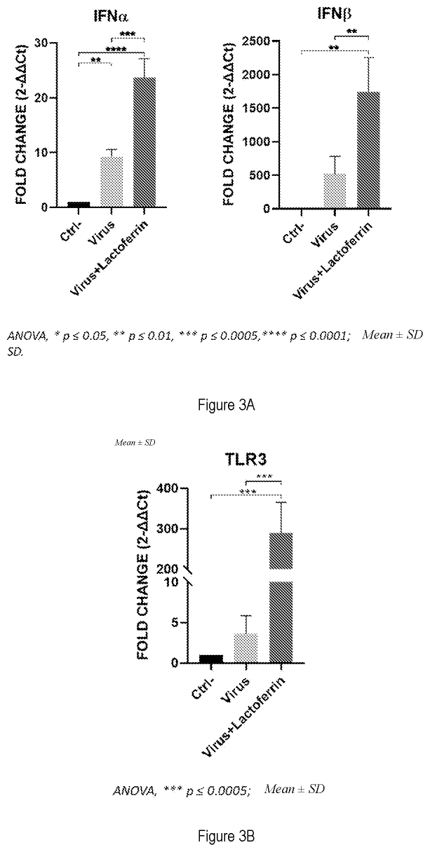

Confluent monolayers of Caco-2 cells were pre-treated or co-treated with lactoferrin (code “L”) with respect to the treatment with SARS-CoV-2 virus according to the scheme reported in FIGS. 2A and 2B; the results obtained are compared with Caco-2 cells treated with the culture medium (control, code “nt”) alone and with Caco-2 cells treated with SARS-CoV-2 virus (code “SARS-CoV-2”) alone.

At the end of the incubation, for each type of treatment, the medium was removed, the total RNA was extracted from the cells, transcribed into cDNA and it was used to carry out quantitative RT-PCR on the following mRNA-transcriptors:
IFN-α and IFN-β, specific for antiviral action;
TLR3 and TLR7, which are specific receptors for innate immunity, which “see” viral RNA and initiate the antiviral response;
MDA5 and MAVS, viral receptors of genes involved in the antiviral response in the cell;
TGF-β, anti-inflammatory marker;
IL-1β and IL-8, pro-inflammatory markers;
TSLP1, cytokine released from intestinal epithelial cells, which has an important effect in the regulation of the anti-inflammatory phenotype of dendritic cells and of macrophages.

All tests and results were conducted three times. Housekeeping gene Rn18S was used as reference. The data were analysed using the ΔΔCt (fold change) method.

Pre-treatment protocol (FIG. 2A): each well was incubated with culture medium alone (control, code “nt”) or treated with lactoferrin at 100 μg/ml. After 3 hours, the medium was removed, replaced with fresh medium containing antibiotics and the monolayers were infected with SARS-CoV-2.

Co-treatment protocol: each well was incubated with culture medium alone (control, code “nt”) or treated with lactoferrin at 100 μg/ml. At the same time the cells were infected with SARS-CoV-2. In these experiments, the medium was removed from all wells after 2 hours and replaced with fresh medium containing antibiotics.

### 2.2. Results

The study showed that lactoferrin is capable of performing both antiviral and anti-inflammatory action in cells infected with SARS-CoV-2 virus, defining lactoferrin suitable for both preventive and curative treatment of COVID-19.

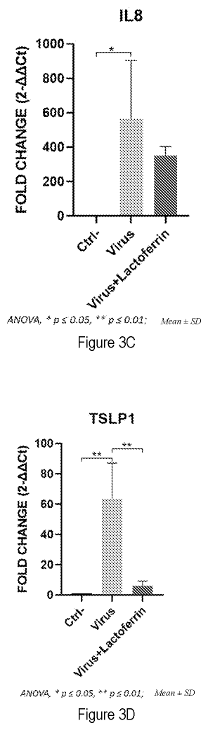

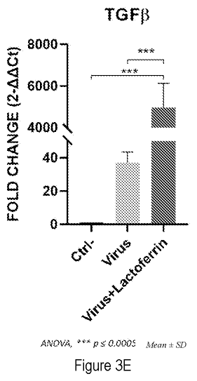

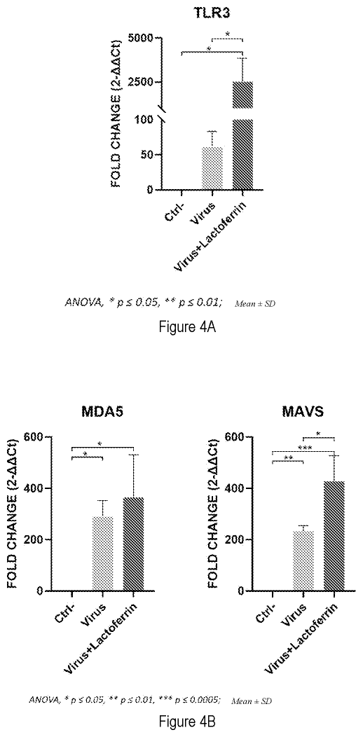

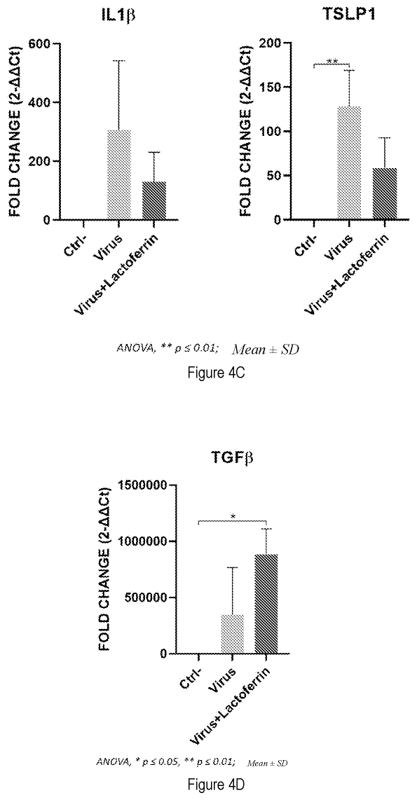

The results reported in FIG. 3 show lactoferrin's:
induction of interferon IFN-α and IFN-β in pre-treatment (FIG. 3A),
induction of TRL3 in pre-treatment (FIG. 3B),
induction of TRL3 in co-treatment (FIG. 4A), and
induction of Mavs and Mda5 in co-treatment (FIG. 4B), involved in viral recognition.

Furthermore, the anti-inflammatory effect of lactoferrin was observed:
reducing the expression of IL-8 in pre-treatment (FIG. 3C) and IL-1β in co-treatment (FIG. 4C),
reducing the expression of TSLP1 in pre-treatment (FIG. 3D) and co-treatment (FIG. 4C), and
increasing the expression of the anti-inflammatory marker TGF-β in pre-treatment (FIG. 3E) and co-treatment (FIG. 4D).

TGF-β was significantly activated in its expression by lactoferrin.

The TLR receptors, based on the cascade of reactions related to the induction of interferons, were activated by lactoferrin, which was particularly active on TLR3 and moderately active on TLR7.

The initiation phase of the antiviral immune response is mediated by MAVS and MDA5; lactoferrin has been found capable of triggering MAVS, although not significantly.

TSLP1 expression was significantly reduced by lactoferrin in the pre-treatment model, demonstrating that lactoferrin contributes toward reducing the virus-induced inflammatory condition.

TSLP1 is a cytokine released from intestinal epithelial cells, it has an important effect in the regulation of the anti-inflammatory phenotype of dendritic cells and of macrophages.

### Conclusion (1) and (2)

The results obtained showed that lactoferrin is capable of positively modulating the antiviral and anti-inflammatory responses, both in the case of pre-treatment and co-treatment experimental protocols, thus being a useful adjuvant in antiviral therapy.

As a matter of fact, it is known from the literature that an increase in pro-inflammatory cytokine expression has been observed in patients with COVID-19, and that the LDH, CRP, PCT serum levels and ferritin have significantly increased in patients with very severe COVID-19 with respect to those less severe. High levels of ferritin and IL-6 are considered predictors of fatality, suggesting that mortality may be due to hyperinflammation caused by viruses.

Thanks to its anti-inflammatory and antiviral properties, lactoferrin (or lactotransferrin) is capable of reducing the inflammatory condition. Lactoferrin, an antimicrobial-action glycoprotein and iron carrier, has been shown to be capable of exerting an anti-inflammatory action against IL-6 in infected/inflamed cells, thus favouring down-regulation of ferritin, key factors in iron homeostasis and inflammatory processes.

1. A composition for use in a method for the treatment of a viral infection,
wherein said composition comprises
(i) lactoferrin; and, optionally,
(ii) at least one acceptable pharmaceutical grade additive and/or excipient; and
wherein said composition is for use through oral route.

2. A composition for use according to claim 1, wherein said composition is for use in a method for the treatment of a viral infection of the respiratory system and of symptoms and/or disorders deriving from or relating to said viral infection; preferably viral infections of the upper respiratory tract and/or of the lower respiratory tract.

3. The composition for use according to claim 1, wherein said viral infection is caused by a virus of the family Coronaviridae, subfamily: Coronavirinae, genus: Betacoronavirus, species: severe acute respiratory syndrome coronavirus, selected from strains: severe acute respiratory syndrome coronavirus (SARS-CoV), severe acute respiratory syndrome coronavirus-2 (SARS-CoV-2 or 2019-nCoV) and responsible for COVID-19 disease, and severe acute respiratory syndrome coronavirus-like (SARS-CoV-like or SL-CoV); preferably SARS-CoV-2.

4. The composition for use according to claim 2, wherein said symptoms and/or disorders deriving from or relating to said viral infection of the respiratory system are selected from: severe acute respiratory syndrome (SARS), respiratory complications, asthma, chronic obstructive pulmonary disease (COPD), bronchitis, emphysema, cystic fibrosis, cough, pertussis, pneumonia, pleurisy, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, kidney failure, loss of appetite, general feeling unwell.

5. The composition for use according to claim 1, wherein the composition is in solid form selected from: tablets, chewable tablets, oral soluble tablets, granules, powder, flakes, soluble powder or granules, oral soluble powder or granules, capsules; or, alternatively, in liquid form selected from: solutions, suspensions, dispersions, emulsions, liquid which can be dispensed in the form of spray, syrups; or, alternatively, in semi-liquid form selected from: soft-gel, gel; preferably in solid form.

6. The composition for use according to claim 1, wherein lactoferrin is in a liposomal form; preferably in a phospholipid-based liposomal form.

7. A method of treating a subject for a viral infection comprising orally administering a composition to the subject, wherein said composition comprises
(i) lactoferrin; and, optionally,
(ii) at least one acceptable pharmaceutical grade additive and/or excipient.

8. The method of claim 7, wherein the patient has a viral infection of the upper respiratory tract and/or of the lower respiratory tract.

9. The method of claim 7, wherein said viral infection is caused by a virus of the family Coronaviridae, subfamily: Coronavirinae, genus: Betacoronavirus, species: severe acute respiratory syndrome coronavirus, selected from strains: severe acute respiratory syndrome coronavirus (SARS-CoV), severe acute respiratory syndrome coronavirus-2 (SARS-CoV-2 or 2019-nCoV) and responsible for COVID-19 disease, and severe acute respiratory syndrome coronavirus-like (SARS-CoV-like or SL-CoV); preferably SARS-CoV-2.

10. The method of claim 7, wherein the patients has severe acute respiratory syndrome (SARS), respiratory complications, asthma, chronic obstructive pulmonary disease (COPD), bronchitis, emphysema, cystic fibrosis, cough, pertussis, pneumonia, pleurisy, bronchiolitis, cold, sinusitis, rhinitis, tracheitis, pharyngitis, laryngitis, acute laryngotracheobronchitis, epiglottitis, bronchiectasis, difficulty breathing, dyspnoea, breathlessness, shortness of breath, fever, fatigue, muscle aches, muscle pain, nasal congestion, runny nose, sore throat, gastrointestinal symptoms, nausea, diarrhoea, kidney failure, loss of appetite, and/or general feeling unwell.

11. The method of claim 7, wherein the composition is in solid form selected from: tablets, chewable tablets, oral soluble tablets, granules, powder, flakes, soluble powder or granules, oral soluble powder or granules, capsules; or, alternatively, in liquid form selected from: solutions, suspensions, dispersions, emulsions, liquid which can be dispensed in the form of spray, syrups; or, alternatively, in semi-liquid form selected from: soft-gel, gel; preferably in solid form.

12. The method of claim 7, wherein the lactoferrin is in a liposomal form; preferably in a phospholipid-based liposomal form.
